(19) United States
(12) Patent Application Publication
(10) Pub. No.: US 2009/0197811 A1
(43) Pub. Date: Aug. 6, 2009

(54) ORALLY ADMINISTERED PEPTIDES TO IMPROVE OR PREVENT HEPATIC FIBROSIS

(75) Inventor: Laurie D. DeLeve, Los Angeles, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 12/174,989

(22) Filed: Jul. 17, 2008

Related U.S. Application Data
(60) Provisional application No. 60/950,694, filed on Jul. 19, 2007.
(60) Provisional application No. 61/060,398, filed on Jun. 10, 2008.

(51) Int. Cl. A61K 38/10

(52) U.S. Cl. 514/13

(57) ABSTRACT

The present invention generally relates to liver disease. More specifically the invention provides compositions and methods, including D-4F, that are useful in treating or preventing hepatic fibrosis.

The present application claims the benefit of the filing date of U.S. Provisional Application No. 60/950,694, filed Jul. 19, 2007 and U.S. Provisional Application No. 61/060,398, filed Jun. 10, 2008, the disclosure of which is incorporated herein by reference in its entirety.

### FUNDING

This invention was made with support by NIH grant DK66423.

### FIELD OF THE INVENTION

The present invention generally relates to fibrosis in the liver. More specifically, the invention provides compositions and methods of using peptides to treat or prevent hepatic fibrosis.

### BACKGROUND OF THE INVENTION

Nonalcoholic fatty liver disease is a chronic liver disease that has been shown to progress to cirrhosis and hepatocellular carcinoma. The prevalence of this disease in the general population has been estimated to be between 3 to 24%. Non-alcoholic fatty liver disease is associated with higher body mass index, insulin resistance, hypertension, high triglycerides, insulin resistance, and/or other factors²³. Much of the morbidity and mortality is a consequence of progression to the advanced stage where there is liver fibrosis.

SR141716, an antagonist of the endocannaboid receptor CB1 has been used in the treatment of non-alcoholic fatty liver disease.¹ CB1 is a pro-fibrotic and its expression is upregulated in cirrhotic liver and its expression on hepatic stellate cells is enhanced upon activation of the stellate cell^{2, 3}. SR141716 has been shown to prevent fibrosis by blocking CB1.²

D-4F has not previously been studied in liver disease. D-4F is a 18 D-amino acid peptide that markedly reduces atherosclerosis in mouse models⁴ and that is currently being investigated in clinical trials for amelioration of atherosclerosis. D-4F is an apolipoprotein A-1 mimetic with antioxidant properties and its lipid-associating structural motifs allow it to interact with phospholipids in cellular membranes and remove oxidized lipids⁵ and it decreases lipoprotein lipid hydroperoxides⁶. Hepatic fibrogenesis is mediated by activation of hepatic stellate cells. Oxidative stress from lipid peroxidation products plays a role in initiating stellate cell activation⁷⁻¹².

C57Bl/6J mice develop obesity, insulin resistant diabetes mellitus, hypercholesterolemia and hypertriglyceridemia when fed a Western diet, a high fat diet formulated to approximate the typical human diet in North America and Europe¹³, We demonstrate that C57Bl/6J mice given 9.5 months of a Western diet develop both steatohepatitis and significant fibrosis. Thus the high saturated fat diet model presented here represents a model of metabolic syndrome with advanced fatty liver disease. We have examined the effect of 2.5 months of treatment with either SR141716 or D-4F in this model.

### SUMMARY OF THE INVENTION

In one embodiment, the invention relates to methods of treating or preventing the development of hepatic fibrosis.

In another embodiment, the invention relates to methods of treating or preventing the progression of hepatic fibrosis.

In a related embodiment, the invention relates to compositions useful for treating or preventing the development of hepatic fibrosis.

In a related embodiment, the invention relates to compositions useful for treating or preventing the progression of hepatic fibrosis.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### DESCRIPTION OF THE FIGURES

FIG. 1. Protocol for the six treatment groups.

FIG. 2. Liver histology. Representative sections of liver after 9.5 months. A. Hematoxylin-eosin stain, B-G: Sirius red stain. A. Liver after 9.5 months of Western diet (group 1) demonstrates steatohepatitis. B. Liver after 9.5 months of Western diet (group 1) demonstrates fibrosis. C. 9.5 months of Western diet with D-4F during last 2.5 months (group 3). D. 9.5 months of Western diet with SR141716 during last 2.5 months (group 2). E. 7 months of Western diet followed by 2.5 months of standard chow (group 4). F. 7 months of Western diet followed by 2.5 months of standard chow plus D-4F (group 6). G. 7 months of Western diet followed by 2.5 months of standard chow plus SR141716 (group 5).

FIG. 3. Morphometric image analysis. The figure demonstrates whole-section-scanning morphometric image analysis of slides stained with Sirius red to determine extent of fibrosis. Control groups: 6-month control (open bar), 6 months of feeding with the Western diet (checked bars). Treatment groups received 7 months of Western diet followed by: 2.5 months of Western diet or chow as indicated (black bar), 2.5 months of D-4F and either Western diet or chow as indicated (hatched bar), or 2.5 months of SR141716 and either Western diet or chow as indicated (dotted bar). Single factor ANOVA for all groups p<0.01; * p<0.005 by least significant difference compared to Western diet; § p<0.001 compared to Western diet and p=0.05 compared to chow for the last 2.5 months by least significant difference; ¶ no significant difference versus 9.5 months Western diet.

FIG. 4. α-Smooth muscle actin staining. A. Control. B. 9.5 months Western diet. C. 7 months Western diet followed by 2.5 months chow. D. 9.5 months Western diet with addition of SR141716 during the last 2.5 months. E. 9.5 months Western diet with addition of D-4F during the last 2.5 months. α-Smooth muscle actin (green) stains activated hepatic stellate cells. Erythrocytes (red) are visible in venules and sinusoids.

FIG. 5. Transmission electron micrograph of the liver of a mouse that received 6 months of the Western diet, illustrating thickened sinusoidal endothelium (E), and a subendothelial basal lamina (arrow) and collagen deposition (CF) in the Space of Disse. S, sinusoid lumen; ×6000 original magnification.

FIG. 6. Scanning electron micrographs of livers from lean mice fed a normal chow diet (A and C) compared to obese mice fed the Western diet for 6 months (B and D). Compare the loss of fenestrae in D vs. C. Note the sinusoids and their pattern distorted by the enlarged, fat-laden parenchymal cells in B vs A. The arrow in D points to the compression of the sinusoid caused by a ballooning hepatocyte containing a large fat droplet. A and B, ×1000 original magnification; C and D, ×8000 original magnification.

FIG. 7. Effect of D-4F on primary murine hepatic stellate cells. Hepatic stellate cells were cultured for 7 days in the presence (B, C, F) or absence (A, E) of D-4F. ASMA staining (ASMA antibody 1:250 dilution) demonstrates that 87% of cells cultured in the absence of D-4F are positive for ASMA (A), whereas none of the stellate cells cultured in the presence of D-4F express ASMA fibres (B). Higher concentrations of ASMA antibody (ASMA antibody 1:50 dilution) demonstrate the characteristic compact morphology of quiescent stellate cells after 7 days culture in the presence of D-4F (C). Oil red O staining of stellate cells after 1 day in culture demonstrates the characteristic lipid droplets (D). Stellate cells cultured for 7 days without D-4F demonstrate few lipid droplets (E), whereas stellate cells cultured in the presence of D-4F maintain significantly more lipid droplets (F).

### DETAILED DESCRIPTION OF THE INVENTION

Nonalcoholic fatty liver disease is a chronic liver disease that has been shown to progress to cirrhosis and hepatocellular carcinoma. The prevalence of this disease in the general population has been estimated to be between 3 to 24%. Non-alcoholic fatty liver disease is associated with higher body mass index, insulin resistance, hypertension, high triglycerides, insulin resistance, and/or other factors²³. Much of the morbidity and mortality is a consequence of progression to the advanced stage in which there is liver fibrosis. Thus, there is a need to develop strategies geared towards the prevention of progression to fibrosis.

The present invention demonstrates that C57Bl/6J mice given 9.5 months of a Western diet develop both steatohepatitis and significant fibrosis. Thus, the high saturated fat diet model presented here represents a model of metabolic syndrome with advanced fatty liver disease. The effect of 2.5 months of treatment with D-4F has been examined in this model.

C57Bl/6J mice were fed a high fat diet for 7 months, followed by 2.5 months treatment with D-4F. D-4F improved hypercholesterolemia and hyperleptinemia without improvement in body weight, steatohepatitis, insulin resistance, or oxidative stress, and yet there was significant prevention of fibrosis. D-4F prevented culture-induced activation of stellate cells in vitro.

As used herein the terms “non-alcoholic fatty liver disease” refer to or describe the physiological condition in mammals that is typically characterized by a range of conditions involving the liver that affect people who drink little or no alcohol but who most commonly have evidence of some or all of the components of the metabolic syndrome: high body mass index, insulin resistance, hypertension, high triglycerides, insulin resistance, hyperglycemia, hypercholesterolemia, hyperleptinemia, and/or other factors. The mildest type is simple fatty liver (steatosis), an accumulation of fat within the liver that usually causes no liver damage. A more advanced stage of steatohepatitis, is associated with liver-damaging inflammation and/or of fibrosis. In some cases, this can progress either to cirrhosis, i.e. progressive, irreversible liver scarring, or to liver cancer.

To practice methods relating to treating or preventing progression of hepatic fibrosis, mammals are given a diet that results in the development of non-alcoholic fatty liver disease with steatohepatitis. At that point, they are given D-4F, except for controls. If the development of fibrosis in the liver is reduced or prevented, then hepatic fibrosis has been treated or prevented.

Compositions relating to the present invention are formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

In one embodiment, the compositions are prepared with carriers that will protect the peptides against rapid elimination from the body, such as a sustained and/or controlled release formulation, including implants and microencapsulated delivery systems. Compositions relating to the present invention, may also comprise siRNAs conjugated to cationic polypeptides, amphipathic compounds, polycations, liposomes or PEGlyated liposomes. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of an active compound calculated to produce the desired therapeutic effect in association with the required pharmaceutical carrier.

The dosage required for treating a subject depends on the choice of the route of administration, the nature of the formulation, the nature of the subject's illness, the subject's size, weight, surface area, age, and sex, other drugs being administered, and the judgment of the attending physician. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Materials and Methods

SR141716 was a kind gift from Sanofi-Aventis, France. SR141716 was dissolved in water with 0.1% Tween and given daily by gavage for 10 weeks. D-4F is an 18-mer of D-amino acids (Ac-D-W-F-K-A-F-Y-D-K-V-A-E-K-F-K-E-A-F-NH2)⁴ (SEQ ID NO: 1) that was commercially synthesized (A & A Laboratories, San Diego Calif.) with 80% purity. D-4F was dissolved in the drinking water at a concentration of 60 μg/ml water for a calculated dose corrected for drug purity of 125 μg D-4F/mouse/day.

The high fat diet used was the RD “Western diet” (Research Diets, D12079B), which contains 41% of calories from fat, 43% calories from carbohydrate, and 17% of calories from protein); this diet was designed to approximate the “typical” human diet of North America and Europe.

### Animal Model.

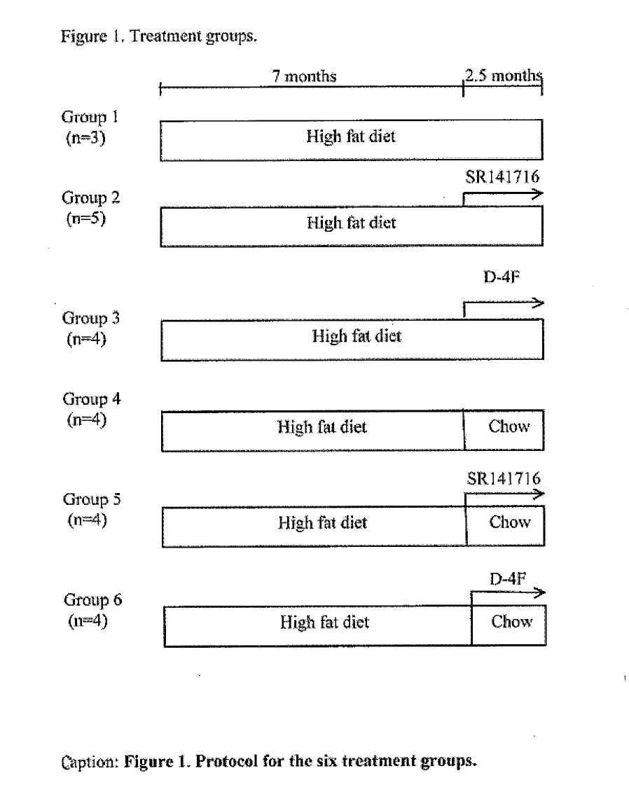

Studies were performed in male C5713L/6J mice (Jackson Laboratories, Bar Harbor Me.). Six groups were studied (n=3-5, see FIG. 1) with 4 mice per group at the outset, except for group 2, which was started with n=5; 1 mouse died in group 1. Groups 1, 2, and 3 received the Western diet for 9.5 months; during the last 2.5 months of the 9.5 months, group 2 received SR141716 10 mg/kg/day i.g., and group 3 received D-4F, 125 μg/mouse/day, in the drinking water. Groups 4, 5, and 6 received the Western diet for 7 months followed by standard chow for 2.5 months; during the 2.5 months on standard chow, group 5 received SR141716 and group 6 received D-4F as described for groups 2 and 3, respectively. Additional control groups were studied: 6-month old C57Bl/6J mice as age-matched controls (n=5) and C57Bl/6J mice that received the Western diet for 6 months (n=6).

All protocols dealing with animals were reviewed and approved by the Animal Care and Use Committees at the University of Southern California and University of Arizona to ensure ethical and humane treatment of the animals. This study followed the guidelines outlined in the NIH “Guide for the Care and Use of Laboratory Animals” (Revised 1985) prepared by the National Academy of Sciences.

### Sirius Red Staining.

Sirius red staining was performed by the Morphology core of the USC-UCLA Research Center for Alcoholic Liver and Pancreatic Diseases. α-Smooth muscle actin.

Sections of formalin-fixed, paraffin-embedded liver, 7 μm thick, were used for α-smooth muscle actin immunofluorescence staining. Slides were deparaffinized in xylene and serially rehydrated in graded ethanol (100-70%). Slides were treated with 0.1 M tris-HCl (pH 7.6)/0.05 Tween-20 for 5 min, incubated with 1 μg/ml proteinase K (Sigma, St Louis Mo.)) for 15 min at room temperature, and then rinsed in water for 10 min. Slides were rinsed in 0.1 M tris-HCl with 2% goat serum for 5 min, incubated with monoclonal anti-α-smooth muscle actin (1:5000, Sigma) for 30 min at room temperature, followed by a FITC-conjugated goat anti-mouse IgG (1:75, Sigma) secondary antibody for 45 min at room temperature. After rinsing in 0.1 M tris-HCl/0.05% Tween-20 for 5 min followed by 0.1 M tris-HCl for 5 min, nuclear staining was performed with DAPI diluted in methanol (1:1000, Sigma) for 5 min. Negative controls were performed by omitting the primary antibody.

Evaluation of Steatosis and/or Fibrosis.

The slides were coded, without the pathologist knowing the specific treatment group that the slides represented. The histology was graded according to a number of histologic features. The degree of fat was graded as to the approximate percentage of hepatocytes containing fat: 0, absent; +/−, less than 5%; 1+, 5-25%; 2+. 26-50%; 3+, 51-75%; 4+, >75%. The fat was also assessed as to whether it was totally macrovesicular, predominantly macrovesicular, totally microvesicular, predominantly microvesicular, or mixed macro- and microvesicular. In addition, the zone was assessed as to whether the fat was perivenular, periportal, diffuse, or focal. Inflammation was graded as absent, scanty (1 to 3 foci/slide), present (scattered, with 4 to 10 foci/slide), present + (scattered, >10 foci/slide but not numerous), and present ++ (numerous). The type of inflammation was graded as totally mononuclear, predominantly mononuclear, totally neutrophilic, predominantly neutrophilic, and mixed mononuclear-neutrophilic. The hydropic change of hepatocytes was graded as absent, mild, and moderate/severe, with the location noted as perivenular, focal or diffuse.

### Whole-Section-Scanning Morphometric Image Analysis.

Tissue sections stained with Sirius red were scanned by a third-generation Automated Cellular Imaging System (ACIS III), a highly automated microscope-based digital imaging system (Carl Zeiss MicroImaging AIS Inc., Aliso Viejo Calif.). The color of the Sirius red staining in the digitized tissue sections was determined and applied to a pre-existing algorithm that provides the area of a target color in μm². The outline of each digitized tissue section was manually traced using the computer's mouse. Areas of large portal tracts and central veins were also outlined manually. The total area (μm²) of each tissue section and of the total area of large portal tracts and central veins were determined by the image analysis system. The area of fibrosis was calculated as [total area stained by Sirius red minus perivascular staining] as a percentage of the size of the tissue section. This method has the advantage of scanning the entire section, rather than measuring fibrosis in randomly chosen fields.

Electron microscopy.

In a separate set of experimental animals (n=3), routine methods were used to prepare liver specimens for transmission (TEM) and scanning (SEM) electron microscopy^{14, 15}. Briefly, the livers were perfused through the portal vein with 0.1 M Na-cacodylate buffer to aid in washing out blood. This was immediately followed with a fixative containing 1.5% glutaraldehyde in 0.1 M cacodylate buffer, pH 7.4. For TEM, minced pieces of liver were further fixed for 2 hrs, then washed in buffer, postfixed with 1% osmium tetroxide in 0.1 M cacodylate buffer at 4° C., dehydrated through graded alcohols, and embedded in Spurr. Thin sections were cut on a Reichert Ultracut microtome, examined and photographed using a Philips CM-12S electron microscope. For SEM, pieces of perfused-fixed livers were dehydrated in a graded ethanol series, fractured, dried with HMDS, sputter-coated with 10 nm gold and examined using a Philips XL30 scanning electron microscope.

### In Vitro Studies.

Murine hepatic stellate cells (HSC) were isolated by collagenase/pronase digestion and Stractan density gradient centrifugation¹⁶. α-Smooth muscle actin (ASMA) staining: HSC were cultured on glass coverslips and fixed with 10% formaldehyde (EM Science, Gibbstown, N.J.) for 20 minutes at room temperature. Cells were incubated with a mouse monoclonal anti-ASMA (1:250 or 1:50, as indicated), followed by a rabbit anti-mouse IgG TRITC conjugate (1:50, Santa Cruz Scientific, Santa Cruz, Calif.). Slides were examined using a Nikon PCM-2000 confocal microscope with a Nikon Eclipse TE300 microscope with a plan Apo 60×/1.4 aperture oil immersion objective, 543-nm laser, and Simple PCI software from the C-Imaging series from Nikon/Compix Inc (Cranberry Township Pa.). Values were obtained by counting the number of ASMA positive cells in 15 randomly selected fields in triplicate. Oil red O staining was performed by the Histology core of the USC Research Center for Liver Disease: slides were stained with oil red O for 10 minutes, rinsed briefly with 70% ethanol, counterstained with Harris' hematoxylin, and then treated with saturated sodium bicarbonate solution.

### Biochemical Assays.

Serum aspartate amino transferase (AST) and alanine aminotransferase (ALT) were measured with the Infinity AST and ALT liquid assay reagent (Thermo Electron Corporation, Louisville Colo., cat #TR70121 and TR71121). Serum triglycerides and cholesterol were measured with the Infinity cholesterol assay reagent (Thermo Electron Corporation, Louisville Colo., cat #TR22321 and TR13315). Serum glucose was measured with the Glucose (HK) assay kit (Sigma, St. Louis, Mo., cat #GAHK-20). Serum insulin was measured with the Mercodia Ultrasensitive mouse insulin ELISA kit (ALPCO Diagnostics, Salem N.H., cat #10 1150-01). The homeostasis model assessment insulin resistance index (HOMA-IR) was calculated as [fasting glucose (mmol/liter)*fasting insulin (microunits/ml)]/22.5^{17, 18}. Serum leptin was measured with the TiterZyme EIA mouse leptin kit (Assay Designs Inc, Ann Arbor Mich., cat #900-019). Serum adiponectin was measured with the mouse adiponectin kit (Biovendor LLC, Candler N.C., cat #RD 293023100). Thiobarbituric acid reactive substance (TBARS) was measured as previously described^{19, 20}; in brief, liver was homogenized and diluted 1:1 in 10% ice cold trichloroacetic acid. 0.5 ml supernatant or of a standard solution of 1,1,3,3-tetraethoxypropane was mixed with 0.5 ml of 0.6% (w/v) 4,6-dihydroxy-2-thiopyrimidine. The solution was boiled for 30 min, cooled and centrifuged. Absorbance was measured at 532 nm with a Shimadzu TV-2101 PC spectrophotometer. To measure hepatic glutathione (GSH), liver was homogenized and diluted 1:1 in 10% ice cold trichloroacetic acid. GSH in the supernatant was measured according to the recycling assay of Tietze²¹.

### Statistical Analysis.

Groups were compared by analysis of variance with post hoc analysis by least significant difference. P<0.05 was considered statistically significant.

### Results

### Western Diet Model

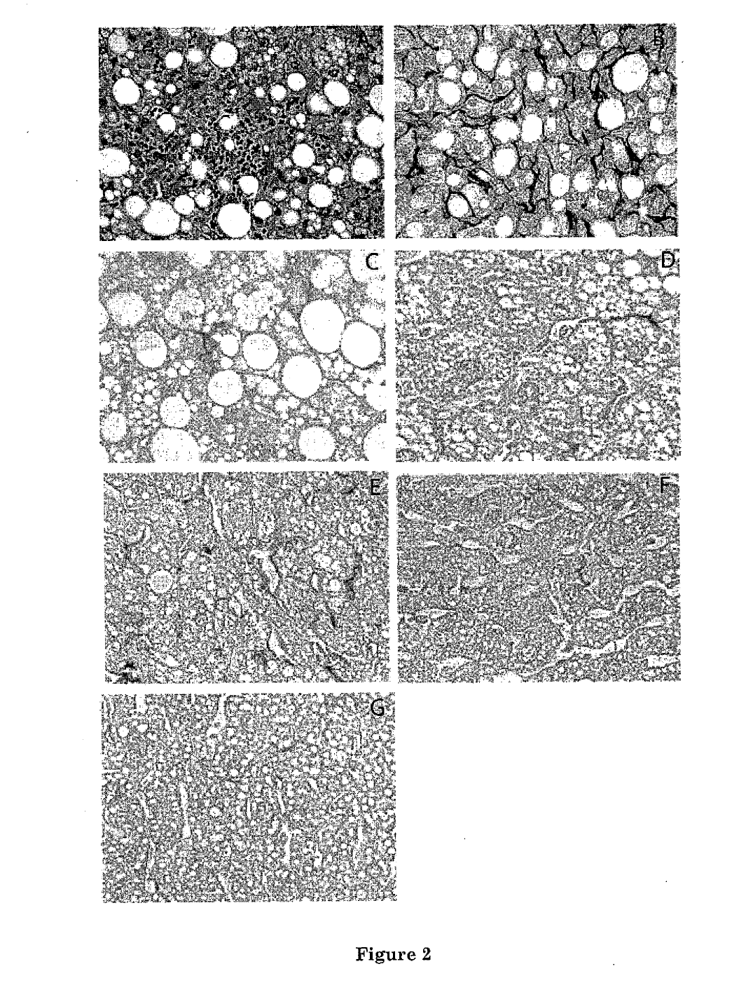

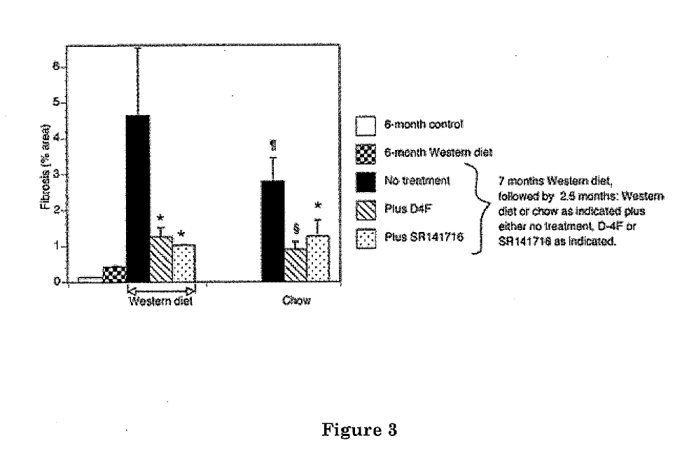

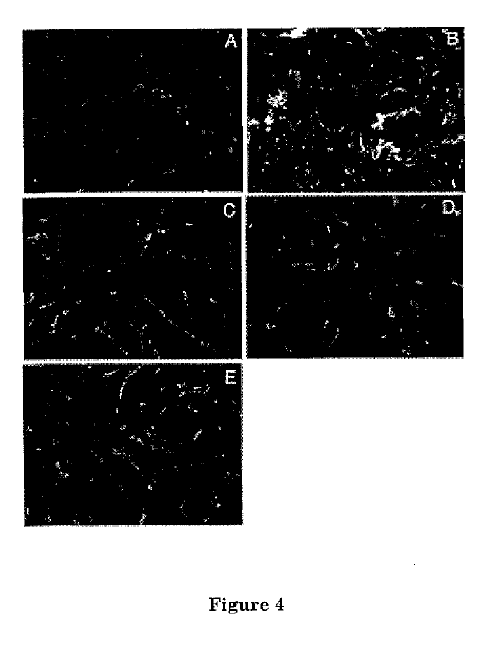

Group 1 (Western diet for 9.5 months) had a 43% increase in body weight, hypercholesterolemia, hyperglycemia, hyperinsulinemia, marked insulin resistance (elevated HOMA-IR), and hyperleptinemia (Table 1). Liver weight was twice that of controls, AST and ALT were elevated 5- to 12-fold, and there was evidence of oxidative stress in the liver with increased tissue TBARS and decreased GSH, Liver histology showed diffusely distributed marked steatosis, foci of mononuclear inflammatory cells, but no hydropic change in the hepatocytes (Table 2, FIG. 2). There was marked fibrosis with 4.6±1.9% of surface area positive for Sirius red staining by whole-section-scanning morphometric image analysis (FIG. 2, FIG. 3). Consistent with the fibrosis, there was increased α-smooth muscle actin staining (FIG. 4).

There were substantial differences in the model after 9.5 months on the Western diet versus 6 months: more pronounced hyperinsulinemia and insulin resistance (HOMA-IR), more evidence of oxidative stress in the liver with higher tissue TBARS and lower hepatic GSH, and higher leptin levels at 9.5 months (Table 1). The major histological difference (Table 2, FIG. 3) was that there was significantly less fibrosis after 6 than after 9.5 months of the Western diet. The surface area of fibrosis by whole-section scanning morphometric image analysis was 0.13±0.01% for the 6-month control, 0.42±0.03% after 6 months on the Western diet, and 4.6±1.9% after 9.5 months Western diet (p<0.01 for 6-month control versus 6 months Western diet; p<0.01 for 6 months versus 9.5 months Western diet).

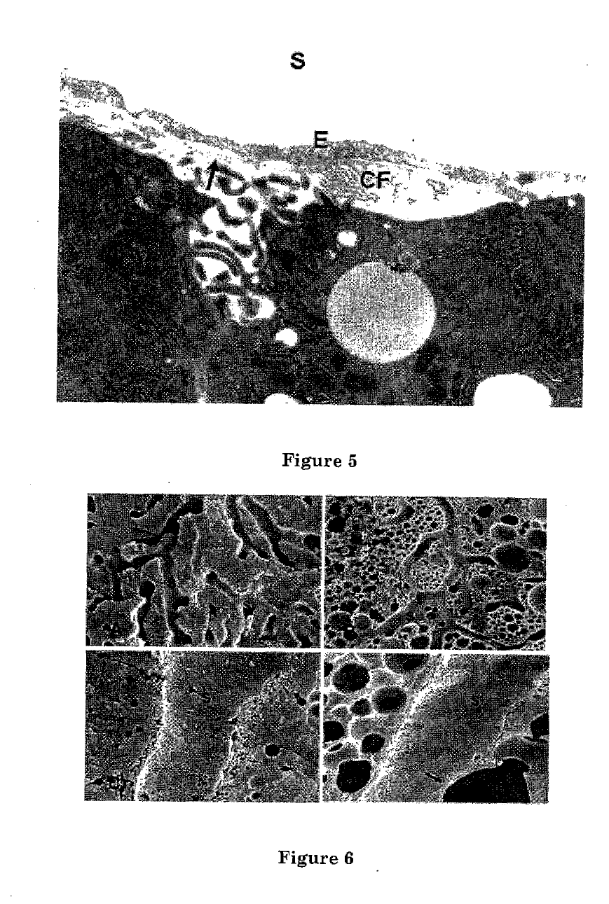

Electron microscopy of mice after 6 months on the Western diet demonstrated early capillarization with defenestration of sinusoidal endothelial cells (FIG. 5, FIG. 6) and formation of a basal lamina (FIG. 6). Scanning electron microscopy demonstrated marked distortion of the sinusoids by fat-laden hepatocytes (FIG. 6).

### D-4F

When mice received the Western diet for 9.5 months with addition of D-4F for the last 2.5 months (group 3), total cholesterol and leptin levels were decreased by half compared to mice on Western diet alone (group 1). There was no improvement in body weight, liver weight, serum transaminases, hyperglycemia, hyperinsulinemia, insulin resistance, or overall hepatic oxidative stress with D-4F (Table 1). Histologically, there was no effect on steatosis and some evidence of a reduction in inflammation (Table 2). Nevertheless, D-4F reduced fibrosis from 4.6±1.9% in group 1 to 1.25% in group 3 (p<0.005; FIGS. 2 and 3).

Comparison of group 4 (switch to chow for the last 2.5 months) with group 6 (switch to chow plus D-4F) demonstrated less fibrosis in the group receiving D-4F: 0.9±0.2% fibrosis in group 6 vs 2.8±0.7% fibrosis in group 4 (p=0.05) and perhaps a small decrease in inflammation (Table 2). No other effect was observed with chow plus D-4F compared to chow alone (Table 1).

Comparison of group 3 (Western diet, D-4F) with group 4 (switch to chow) demonstrated that group 4 had greater improvement in body and liver weight, steatohepatitis (AST, ALT, histologic steatosis), hypercholesterolemia, hyperglycemia, insulin resistance, hyperleptinemia, and overall hepatic oxidative stress (TBARS, GSH). Moreover, group 3 had a significant reduction in progression of fibrosis and group 4 did not (FIGS. 2 and 3).

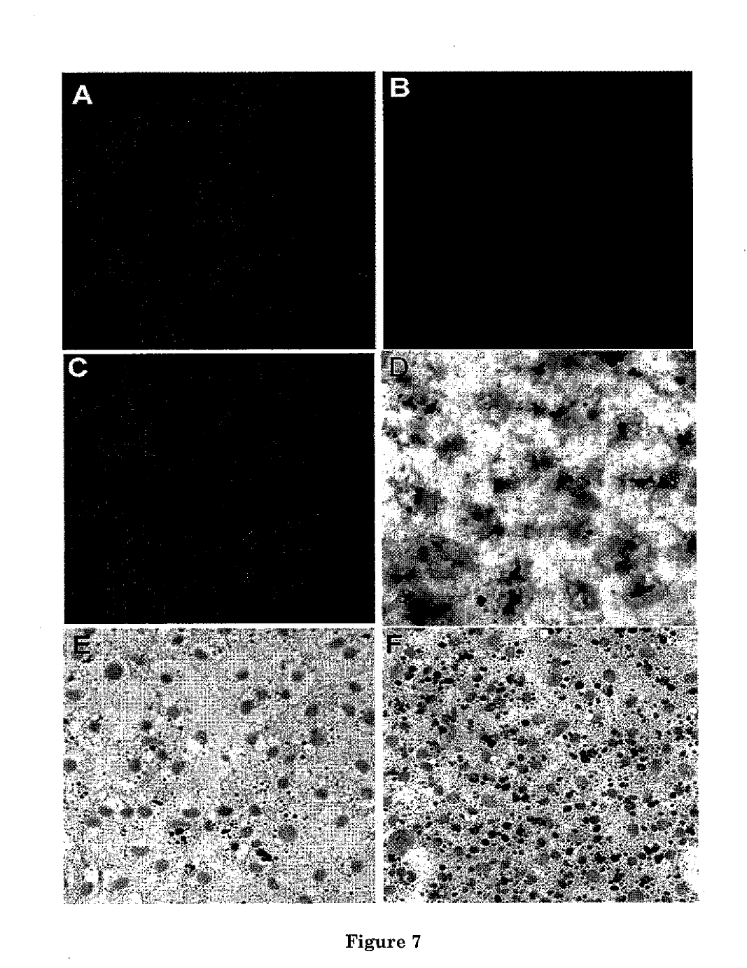

To examine the mechanism of action of D-4F, murine hepatic stellate cells (HSC) were cultured on plastic for 7 days in the absence or presence of 10 μg/ml D-4F. Without D-4F, 87±2% of cells expressed α-smooth muscle actin (ASMA) after 7 days as assessed by confocal microscopy. In contrast, in the presence of D-4F 0% of HSC expressed ASMA after 7 days (see FIG. 7). HSC cultured in the presence of D-4F also maintained cytoplasmic fat droplets, in contrast to USC cultured without D-4F (see FIG. 7). This decrease in stellate cell activation in vitro is consistent with the decrease in activated stellate cells seen in liver sections from mice on the Western diet treated with D-4F (FIG. 4E). The lack of stellate cell activation in vitro demonstrates that the effect of D-4F is not related to factors specific to non-alcohol fatty liver disease, but is a direct effect on stellate cells.

### Discussion

C57Bl/6J mice given 9.5 months of a high saturated fat diet formulated to approximate the typical human diet in North America and Europe 13 developed non-alcoholic steatohepatitis with significant fibrosis and signs of the metabolic syndrome (obesity, insulin resistance, hyperglycemia, and hypercholesterolemia). This provides a model to examine interventions that prevent progression of fibrosis and to correlate the degree of fibrosis with features of steatohepatitis and the metabolic syndrome.

In mice remaining on the Western diet and treated with D-4F there was improvement in hypercholesterolemia and hyperleptinemia, no improvement in body weight, steatohepatitis, insulin resistance, or oxidative stress, and yet there was significant prevention of fibrosis.

From a clinical perspective, the most important comparison may be the effect of switching to chow versus the use of D-4F with continuation of the Western diet, i.e. comparing the effect of switching to a healthier diet with the effect of medication and a continued high fat diet.

D-4F prevented progression of fibrosis to a comparable degree to SR141716 in mice continued on the Western diet. D-4F had no effect on steatohepatitis, and no effect on overall oxidative stress in liver tissue. The only extrahepatic effects of D-4F that were observed were reduction in leptin and cholesterol in the mice continued on the Western diet. However the combination of D-4F and a switch to chow reduced fibrosis more than chow alone and in this group D-4F had no additive effect on cholesterol and leptin compared to the switch to chow alone. The in vitro studies demonstrated that D-4F prevented hepatic stellate cell activation in vitro. Thus D-4F is a general anti-fibrotic agent by prevention of hepatic stellate cell activation rather than an anti-fibrotic agent only in the setting of non-alcoholic fatty liver disease.

In summary, both SR141716 and D-4F prevented progression of fibrosis after onset of steatohepatitis, i.e. a situation comparable to a common clinical scenario, and either compound therefore has the potential to be of clinical benefit. SR141716 promoted weight loss, improved steatohepatitis, decreased insulin resistance, decreased leptin levels and prevented fibrosis through antagonism of CB1. D-4F1 did not alter steatohepatitis, but prevented stellate cell activation in vitro and is therefore a more general anti-fibrotic agent.

TABLE 1
Biochemical findings and liver and body weights.

| | | 6-mo | 7 months Western diet followed by 2.5 months of | | |
|---|---|---|---|---|---|
| | 6-mo | Western | Western | Western | Western |
| | control | diet | diet | diet + D4F | diet + SR141716 |
| Body weight (g) | 28.1 ± 1.1 | 45.9 ± 1.2 | 40 ± 5.2 | 39.5 ± 0.8 | 29.1 ± 2.0 |
| Liver weight (g) | 1.34 ± 0.05 | 3.7 ± 0.2 | 2.74 ± 0.67 | 2.42 ± 0.04 | 1.54 ± 0.1 |
| AST (U/L) | 54.7 ± 10.0 | 250.7 ± 27.1 | 268 ± 29.6 | 211.9 ± 19.1 | 109.6 ± 10.4 |
| ALT (U/L) | 18.8 ± 4.0 | 300.0 ± 37.1 | 235.2 ± 23.5 | 277.3 ± 18.2 | 54.8 ± 12.4 |
| Cholesterol (mg/dl) | 900 ± 5.1 | 274.1 ± 22.1 | 288.0 ± 5.4 | 146.8 ± 2.1 | 112.2 ± 13.7 |
| Triglycerides (mg/dl) | 50.7 ± 7.7 | 32.4 ± 5.6 | 38.0 ± 4.4 | 40.0 ± 6.7 | 33.1 ± 2.2 |
| Glucose (mg/dl) | 65.0 ± 1.2 | 261.0 ± 24.2 | 270.2 ± 10.0 | 231.2 ± 18.0 | 136.3 ± 29.2 |
| Insulin (ng/ml) | 0.47 ± 0.06 | 2.21 ± 0.09 | 3.27 ± 3.0 | 3.7 ± 1.1 | 0.81 ± 0.15 |
| HOMA-IR | 2.2 ± 0.3 | 40.9 ± 4.2 | 62.8 ± 5.1 | 63.3 ± 23.5 | 8.8 ± 3.6 |
| TBARS (nmol/g tissue) | 18.7 ± 1.2 | 16.4 ± 0.4 | 27.1 ± 4.0 | 29.4 ± 2.4 | 17.1 ± 0.7 |
| GSH (nmole/mg protein) | 91.8 ± 3.8 | 71.2 ± 3.9 | 39.6 ± 4.8 | 50.1 ± 5.8 | 68.7 ± 2.0 |
| Leptin (ng/ml) | 1.89 ± 0.29 | 24.0 ± 2.2 | 32.62 ± 1.86 | 17.49 ± 1.41 | 2.36 ± 1.05 |
| Adiponectin (μg/ml) | 22.9 ± 0.3 | 31.1 ± 1.9 | 17.3 ± 0.8 | 15.3 ± 1.0 | 18.4 ± 1.8 |

| | | 6-mo | | | |
|---|---|---|---|---|---|
| | 6-mo | Western | | Chow + | Chow + |
| | control | diet | Chow | D4F | SR141716 |
| Body weight (g) | 28.1 ± 1.1 | 45.9 ± 1.2 | 34.6 ± 1.2 | 35.9 ± 3.2 | 30.2 ± 1.1 |
| Liver Weight (g) | 1.34 ± 0.05 | 3.7 ± 0.2 | 1.54 ± 0.09 | 1.67 ± 0.19 | 1.39 ± 0.1 |
| AST (U/L) | 54.7 ± 10.0 | 250.7 ± 27.1 | 107.2 ± 20.5 | 104.2 ± 22.4 | 69.1 ± 5.5 |
| ALT (U/L) | 18.8 ± 4.0 | 300.0 ± 37.1 | 44.0 ± 4.0 | 69.1 ± 21.5 | 59.1 ± 11.4 |
| Cholesterol (mg/dl) | 90.0 ± 5.1 | 274.1 ± 22.1 | 108.6 ± 4.7 | 120.8 ± 2.9 | 105.3 ± 4.7 |
| Triglycerides (mg/dl) | 50.7 ± 7.7 | 32.4 ± 5.6 | 36.2 ± 6.6 | 49.1 ± 1.4 | 47.0 ± 2.2 |
| Glucose (mg/dl) | 65.0 ± 1.2 | 261.0 ± 24.2 | 137.2 ± 18.6 | 159.2 ± 2.4 | 96.2 ± 3.0 |
| Insulin (ng/ml) | 0.47 ± 0.06 | 2.21 ± 0.09 | 1.78 ± 0.39 | 1.46 ± 0.2 | 0.89 ± 0.27 |
| HOMA-IR | 2.2 ± 0.3 | 40.9 ± 4.2 | 18.1 ± 5.1 | 16.5 ± 2.2 | 6.1 ± 2.0 |
| TBARS (nmol/g tissue) | 18.7 ± 1.2 | 16.4 ± 0.4 | 20.4 ± 0.3 | 20.0 ± 0.6 | 18.7 ± 1.09 |
| GSH (nmole/mg protein) | 91.8 ± 3.8 | 71.2 ± 3.9 | 85.1 ± 3.8 | 82.8 ± 5.2 | 73.8 ± 2.2 |
| Leptin (ng/ml) | 1.89 ± 0.29 | 24.0 ± 2.2 | 9.36 ± 0.76 | 8.70 ± 1.43 | 1.95 ± 0.72 |
| Adiponectin (μg/ml) | 22.9 ± 0.3 | 31.1 ± 1.9 | 19.3 ± 1.1 | 18.52 ± 1.5 | 16.5 ± 2.0 |

TABLE 2
Assessment of steatosis and inflammation

| 7 months of Western | | | Inflam- |
|---|---|---|---|
| diet followed | Fatty Change | | matory |
| by 2.5 months of | Degree of fat | Type¹ | Clusters² |
| Western diet | 4 | Macrovesicular | 4 |
| | 4 | Mixed | 3 |
| | 1 | Macrovesicular | 4 |
| Western diet + D4F | 4 | Macrovesicular | 3 |
| | 4 | Macrovesicular | 3 |
| | 4 | Macrovesicular | 2 |
| | 4 | Macrovesicular | 3 |
| Western diet + SR141716 | 4 | Microvesicular | 1 |
| | 3 | Mixed | 2 |
| | ± | Macrovesicular | 2 |
| | 1 | Mixed | 2 |
| | 4 | Microvesicular | 2 |
| Chow | 2 | Mixed | 2 |
| | 1 | Mixed | 3 |
| | 1 | Microvesicular | 2 |
| | 1 | Mixed | 3 |
| Chow + D4F | 2 | Mixed | 1 |
| | 0 | — | 2 |
| | 2 | Mixed | 2 |
| | 3 | Mixed | 2 |
| Chow + SR141716 | 3 | Microvesicular | 2 |
| | 2 | Microvesicular | 2 |
| | 1 | Microvesicular | 2 |
| | 3 | Microvesicular | 1 |

¹The type of fatty change listed indicates the predominant type present. Steatosis was diffusely distributed in mice that remained on the Western diet with or without D-4F, and perivenular in mice that switched to chow with or without D-4F; steatosis was diffuse or perivenular in mice on SR141716.
²Inflammatory clusters were composed of mononuclear cells.

### REFERENCES

The following references are cited herein. The entire disclosure of each reference is relied upon and incorporated by reference herein.
1. Osei-Hyiaman D, DePetrillo M, Pacher P, Liu J, Radaeva S, Batkai S, Harvey-White J, Mackie K, Offertaler L, Wang L, Kunos G: Endocannabinoid activation at hepatic CB1 receptors stimulates fatty acid synthesis and contributes to diet-induced obesity, Journal of Clinical Investigation 2005, 115:1298-1305
2. Teixeira-Clerc F, Julien B, Grenard P, Tran Van Nhieu J, Deveaux V, Li L, Serriere-Lanneau V, Ledent C, Mallat A, Lotersztajn S: CB1 cannabinoid receptor antagonism: a new strategy for the treatment of liver fibrosis.[see comment], Nature Medicine 2006, 12:671-676
3. Julien B, Grenard P, Teixeira-Clerc F, Van Nhieu J T, Li L, Karsak M, Zimmer A, Mallat A, Lotersztajn S: Antifibrogenic role of the cannabinoid receptor CB2 in the liver, Gastroenterology 2005, 128:742-755
4. Navab M, Anantharamaiah G M, Hama S, Garber D W, Chaddha M, Hough G, Lallone R, Fogelman A M: Oral administration of an Apo A-I mimetic Peptide synthesized from D-amino acids dramatically reduces atherosclerosis in mice independent of plasma cholesterol, Circulation 2002, 105:290-292
5. Anantharamaiah G M, Mishra V K, Garber D W, Datta G, Handattu S P, Palgunachari M N, Chaddha M, Navab M, Ready S T, Segrest J P, Fogelman A M, Anantharamaiah G M, Mishra V K, Garber D W, Datta G, Handattu S P, Palgunachari M N, Chaddha M, Navab M, Reddy S T, Segrest J P, Fogelman A M: Structural requirements for antioxidative and anti-inflammatory properties of apolipoprotein A-I mimetic peptides, Journal of Lipid Research 2007, 48:1915-1923
6. Navab M, Anantharamaiah G M, Reddy S T, Hama S, Hough G, Grijalva V R, Wagner A C, Frank J S, Datta G, Garber D, Fogelman A M: Oral D-4F causes formation of pre-beta high-density lipoprotein and improves high-density lipoprotein-mediated cholesterol efflux and reverse cholesterol transport from macrophages in apolipoprotein E-null mice, Circulation 2004, 109:3215-3220
7. MacDonald G A, Bridle K R, Ward P J, Walker N I, Houglum K, George D K, Smith J L, Powell L W, Crawford D H, Ramm G A: Lipid peroxidation in hepatic steatosis in humans is associated with hepatic fibrosis and occurs predominately in acinar zone 3 Journal of Gastroenterology & Hepatology 2001, 16:599-606
8. Nieto N, Friedman St, Cederbaum A I: Cytochrome P450 2E1-derived reactive oxygen species mediate paracrine stimulation of collagen I protein synthesis by hepatic stellate cells, Journal of Biological Chemistry 2002, 277:9853-9864
9. Poli G: Pathogenesis of liver fibrosis: role of oxidative stress, Molecular Aspects of Medicine 2000, 21:49-98
10. Schneiderhan W, Schmid-Kotsas A, Zhao J, Grunert A, Nussler A, Weidenbach H, Menke A, Schmid R M, Adler G, Bachem M G: Oxidized low-density lipoproteins bind to the scavenger receptor, CD36, of hepatic stellate cells and stimulate extracellular matrix synthesis, Hepatology 2001, 34:729-737
11. Svegliati Baroni G, D'Ambrosio L, Ferretti G, Casini A, Di Sario A, Salzano R, Ridolfi F, Saccomanno S, Jezequel A M, Benedetti A: Fibrogenic effect of oxidative stress on rat hepatic stellate cells, Hepatology 1998, 27:720-726
12. Tsukamoto H, Rippe R, Niemela O., Lin M: Roles of oxidative stress in activation of Kupffer and Ito cells in liver fibrogenesis, Journal of Gastroenterology & Hepatology 1995, 10 Suppl 1:550-53
13. Merat S, Casanada F, Sutphin M, Palinski W, Reaven P D: Western-type diets induce insulin resistance and hyperinsulinemia in LDL receptor-deficient mice but do not increase aortic atherosclerosis compared with normoinsulinemic mice in which similar plasma cholesterol levels are achieved by a fructose-rich diet, Arteriosclerosis, Thrombosis & Vascular Biology 1999, 19:1223-1230
14. McCuskey R S: Microscopic methods for studying the microvasculature of internal organs. Edited by Baker C H, Nastuk W F. New York, Academic Press, 1986, p. pp. 247-264
15. McCuskey R S, McCuskey P A: The fine structure and function of Kupffer cells, Journal of Electron Microscopic Techniques 1990, 14:237-246
16. Tsukamoto H, Towner S J, Ciofalo L M, French S W: Ethanol-induced liver fibrosis in rats fed high fat diet, Hepatology 1986, 6:814-822
17. Xia Z, Sniderman A D, Cianflone K: Acylation-stimulating Protein (ASP) Deficiency Induces Obesity Resistance and Increased Energy Expenditure in ob/ob Mice, J. Biol. Chem. 2002, 277:45874-45879
18. Matthews D R, Hosker J P, Rudenski A S, Naylor B A, Treacher D F, Turner R C: Homeostasis model assessment: insulin resistance and beta-cell function from fasting plasma glucose and insulin concentrations in man. [see comment], Diabetologia 1985, 28:412-419
19. Wong S H Y, Knight J A, Hopfer S M, Zaharia O, Leach C N, Jr., Sunderman F W, Jr.: Lipoperoxides in plasma as measured by liquid-chromatographic separation of malondialdehyde-thiobarbituric acid adduct, Clinical Chemistry 1987,
20. Wallin B, Rosengren B, Shertzer H G, Camejo G: Lipoprotein Oxidation and Measurement of Thiobarbituric Acid Reacting Substances Formation in a Single Microtiter Plate—Its Use for Evaluation of Antioxidants, Analytical Biochemistry 1993, 208:10-15
21. Tietze F: Enzymic method for quantitative determination of nanogram amounts of total and oxidised glutathione: applications to mammalian blood and other tissues, Analytical Biochemistry 1969, 27:502-522
22. Saxena N K, Saliba G, Floyd J J, Anania F A: Leptin induces increased alpha2 (I) collagen gene expression in cultured rat hepatic stellate cells, Journal of Cellular Biochemistry 2003, 89:311-320
23. Clark J M. The epidemiology of nonalcoholic fatty liver disease in adults. Journal of Clinical Gastroenterology 2006; 40:S5-10.

What is claimed:

1. A method of treatment to prevent the development of hepatic fibrosis in a subject in need thereof, said method comprises administering to said subject an effective amount of D-4F.

2. The method according to claim 1, wherein said D-4F comprises SEQ ID NO: 1.

3. The method according to claim 1, wherein said D-4F is administered orally, topically, by sustained-release subcutaneous formulation, or by intravenous infusion.

4. The method according to claim 1, wherein said D-4F is administered orally.

5. The method according to claim 1, wherein said subject is a mammal.

6. The method of claim 1, wherein said subject has one or more abnormalities consistent with or signs of liver disease.

7. The method according to claim 1, wherein said subject is at risk for developing non-alcoholic fatty liver disease.

8. A method of treatment to prevent the progression of liver disease to an advanced stage in a subject in need thereof said method comprises administering to said subject an effective amount of D-4F.

9. The method according to claim 8, wherein said D-4F comprises SEQ ID NO: 1.

10. The method according to claim 8, wherein said D-4F is administered orally, topically, by sustained-release subcutaneous formulation, or by intravenous infusion.

11. The method according to claim 8, wherein said D-4F is administered orally.

12. The method according to claim 8, wherein said subject is a mammal.

13. The method according to claim 8, wherein said subject has non-alcoholic fatty liver disease.

14. A method of preventing the progression of hepatic fibrosis in a subject in need thereof, said method comprises administering to said subject an effective amount of a D-4F.

15. The method according to claim 9, wherein said D-4F comprises SEQ ID NO: 1.

16. The method according to claim 9, wherein said D-4F is administered orally, topically, by sustained-release subcutaneous formulation or by intravenous infusion.

17. The method according to claim 9, wherein said D-4F is administered orally.

18. The method according to claim 9, wherein said subject is a mammal.
